(19) United States
(12) Patent Application Publication
(10) Pub. No.: US 2002/0165555 A1
(43) Pub. Date: Nov. 7, 2002

(54) Shape memory surgical polypectomy tool

(76) Inventors: Barry L. Stein, Cote St-Luc (CA); Alvin Marion Post, Tucson, AZ (US)

Correspondence Address: DOWELL & DOWELL, P.C., Suite 309, 1215 Jefferson Davis Highway, Arlington, VA, 22202-3124, US

(21) Appl. No.: 10/116,158

(22) Filed: Apr. 5, 2002

Related U.S. Application Data
(60) Provisional application No. 60/281,413, filed on Apr. 5, 2001.

(51) Int. Cl. A61B 17/24

(52) U.S. Cl. 606/113

(57) ABSTRACT

The present invention relates to surgical instruments and more specifically to surgical instruments for removing polyps and growths from within a patient's body. The surgical instrument of the present invention comprises an electrically conductive probe and a shape memory alloy filament attached to the working end of the electrically conductive probe. The shape memory alloy has a first working position and a second working position and is capable of acquiring the second working position in response to the passage of electrical current flowing through, and elevating the temperature of the filament.

### FIELD OF THE INVENTION

The present invention relates to the field of surgical tools and more specifically, to surgical tools that use strands or filaments of shape memory alloy to remove polyps and growths from within a patient's body.

### BACKGROUND OF THE INVENTION

Surgical tools for removing polyps and growths from the colon and other areas of the human body are well known in the art. Examples of such surgical tools are described in U.S. Pat. No. 6,015,415, U.S. Pat. No. 5,908,429, and Russian Patent 5004687.

Specifically, U.S. Pat. No. 6,015,415 describes a surgical snare tool for removing polyps that comprises a handle assembly, a tubular sheath, a flexible probe and a closed snare-loop that is located at the working end of the flexible probe. In operation, the handle assembly is used to maneuver the snare-loop within a patient's body in order to loop the snare-loop around a designated polyp. The snare-loop is built so that as it exits the tubular sheath and extends parallel to the tubular sheath but non-axially, thereby allowing the snare-loop to get closer to the base of the polyp than if it were aligned axially with the sheath.

Once the snare-loop has been looped around the designated polyp, the user operates the hand assembly in such a way as to tighten the snare-loop securely around the designated polyp. Once the loop is securely tightened, a cautery current is transmitted through the flexible probe to the snare-loop, so that the snare-loop can burn through the designated polyp.

The drawback of the surgical snare tool described in U.S. Pat. No. 6,015,415 is that the snare-loop is a noose shaped device that needs to be located around the polyp to be removed. During surgery it is not always possible to place the loop around the polyp. Polyps may be very long, or positioned in such a way that it is difficult if not impossible to position the loop around them. In these cases more invasive surgery, and an excessive amount of time, is required in order to remove the polyp.

Against this background it is clear that there is a need in the industry for a wider range of improved surgical polypectomy tools that are capable of easily and efficiently capturing and removing polyps from within a patient's body.

### SUMMARY OF THE INVENTION

As embodied and broadly described herein, the present invention provides a surgical tool for removing growths from within a patient's body. The surgical tool comprises an electrically conductive probe that has a working end that is adapted to be inserted into a patient's body and a shape memory alloy filament that is attached to the working end of the electrically conductive probe. The shape memory alloy filament has a first working position and a second working position. In the first working position, the shape memory alloy filament is in a position that enables it to be inserted into a patient's body and placed next to a growth designated for removal. In the second working position, the shape memory alloy filament forms a bend around a polyp or other growth.

The shape memory alloy filament is capable of transitioning from the first working position to the second working position in response to the passage of electrical current therethrough that elevates the temperature of the filament. As the electrical current passes through the filament, the filament becomes hot, which allows the filament to excise the polyp or other growth.

As further embodied and broadly described herein, the present invention provides a process for removing a growth from within a patient's body. The process comprises providing a shape memory alloy filament that has a first working position and a second working position. In the first working position the shape memory alloy filament is in a condition that enables it to be inserted into a patient's body and placed next to a growth. In the second working position, the shape memory alloy filament forms a bend around the growth. The shape memory alloy filament is able to transition from the first working position to the second working position in response to the passage of electrical current therethrough that elevates the temperature of the shape memory alloy filament. As the electrical current passes through the filament, the filament becomes hot, which allows the filament to excise the polyp or other growth.

The process further comprises inserting the shape memory alloy filament into the patient's body while the shape memory alloy filament is in the first working position, positioning the shape memory alloy filament while it is in the first working position next to the growth and applying an electrical current through the shape memory alloy filament for transitioning the shape memory alloy filament into the second working position wherein the shape memory alloy filament forms a bend around the growth. Finally, the process comprises excising the growth with the shape memory alloy filament.

As still further embodied and broadly described herein, the present invention provides a method for setting at least one working position of a shape memory alloy filament for use in excising a growth from within a patient's body. The method comprises forming the shape memory alloy filament into a bend, heating the shaped memory alloy while in the formed bend, and quenching the shape memory alloy filament while in the formed bend.

### BRIEF DESCRIPTION OF THE DRAWINGS

FIG. 1 is top plan view of the surgical tool according to a specific embodiment of the present invention;

FIG. 2 is an enlarged view of the working end of the surgical tool of FIG. 1 shown in the straight position, which the curved position indicated by dotted lines;

FIG. 3 is the working end of the surgical tool of FIG. 2 positioned next to a polyp;

FIG. 4 is the working end of the surgical tool shown in FIG. 3 around the polyp;

FIG. 5 is an expanded view of the working end of the surgical tool according to an alternative embodiment of the invention;

FIG. 6 is an expanded view of the working end of the surgical tool according to a further alternative embodiment of the invention;

FIG. 7 is an expanded view of the working end of the surgical tool according to a still further alternative embodiment of the invention.

### DETAILED DESCRIPTION

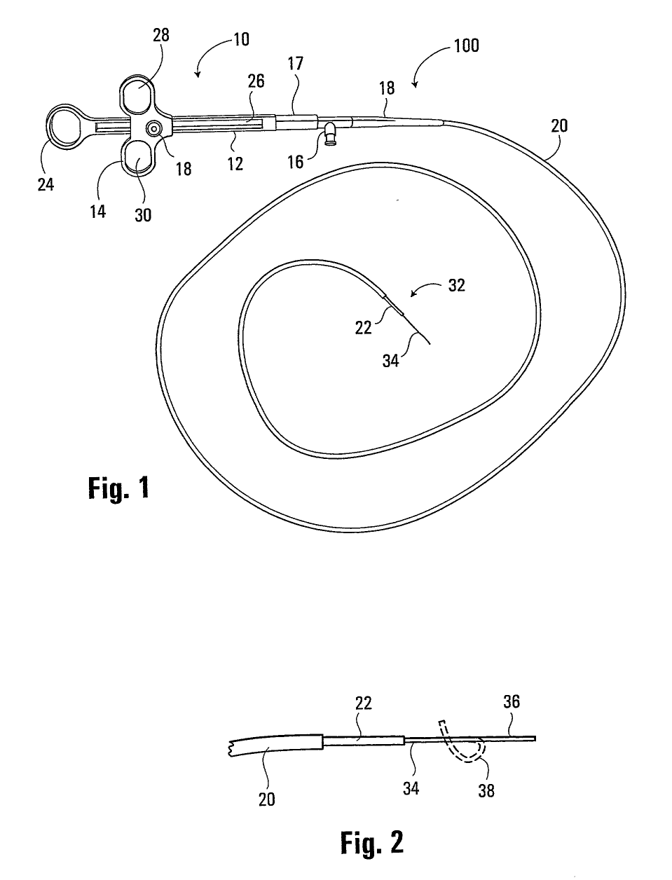

Shown in FIG. 1 is a shape memory surgical tool 100 in accordance with a specific embodiment of the invention that is able to excise growths and polyps from within a patient's body. Surgical tool 100 has a handle assembly 10, a long flexible electrically insulating sheath 20 and an electrically conductive flexible and extendable probe 22. Handle assembly 10 includes a frame section 12 and a finger section 14. Frame section 12 contains a thumb hold 24 and a track 26. Finger section 14 includes two finger holds 28 and 30, and is adapted to slide from one end of frame section 12 to the other, along track 26. Handle assembly 10 further includes a terminal 18 for attachment to an electrical power supply unit. The electrical power supply unit allows the electrical current supplied to terminal 18 to be varied. The lower portion of handle assembly 10 comprises an irrigation port 16 and a tubular section 17, both of which are axially rotatable in relation to frame 12, thereby permitting rotation of sheath 20.

Flexible sheath 20 is connected to the bottom of frame section 12. A shorter more rigid sheath 19 is also connected to the bottom of frame section 12 and is positioned over sheath 20, thereby protecting sheath 20 from bending too sharply at its connection to frame section 12. Probe 22 is made of an electrically conductive material, and fits slidably within sheath 20. Probe 22 is attached to movable finger section 14 of hand assembly 10, such that as finger section 14 moves up and down along track 26 of frame section 12, probe 22 moves in and out of sheath 20 at working end 32. When movable finger section 14 is at the base of frame 12, probe 22 is at its most extended position outside sheath 20. When movable finger section 12 is at the position closest to thumb hold 24, then probe 22 is retracted as far inside sheath 20 as possible. Although flexible sheath 20 has been described as being connected to frame section 12, and probe 22 has been described as being connected to finger section 14, it is within the scope of the invention for flexible sheath 20 to be connected to finger section 14, and probe 22 to be connected to frame section 12.

At working end 32, a filament or strand of shape memory alloy is welded, or attached mechanically by any suitable means to the working end of probe 22. As examples of non-limiting means of attachment, filament 34 can be welded, brazed, silver soldered or swaged in place. In a preferred embodiment, filament 34 of shape memory alloy is nickel titanium(Ni—Ti) with heat activated shape memory properties. As can be seen in FIG. 2, filament 34 of Ni—Ti has two working positions, namely, a straight working position 36, and a curved working position 38. In the curved working position 38, filament 34 is able to encircle a polyp to be excised. As a non-limiting example of a curved working position, filament 34 is bent into a continuous circular loop. However, in alternate embodiments of a curved working position, filament 34 forms a series of straight segments joined by bends having angles of less than 180 degrees. In such positions, filament 34 can be in the form of a triangle, square, etc.

In a very specific and non-limiting example, filament 34 is a Ni—Ti wire with a diameter of 0.015 inches and a phase transition temperature of approximately 70 degrees Celsius. Alternatively, filament 34 may be of a thicker diameter, which will provide more strength, however filaments having a thicker diameters will not form as tight a curved working position as a filament having a thinner diameter.

In order to achieve the heat-activated curved working position 38, filament 34 is pre-treated. The pre-treatment process includes forming filament 34 such that it includes at least one bend, heating filament 34, and quenching filament 34 in cold water. After pre-treatment, filament 34 is straightened back into its straight working position 36 in preparation for surgery. In order to return to the curved working position 38, filament 34 is heated. It should be expressly understood that other manufacturing techniques are possible and are within the scope of the present invention.

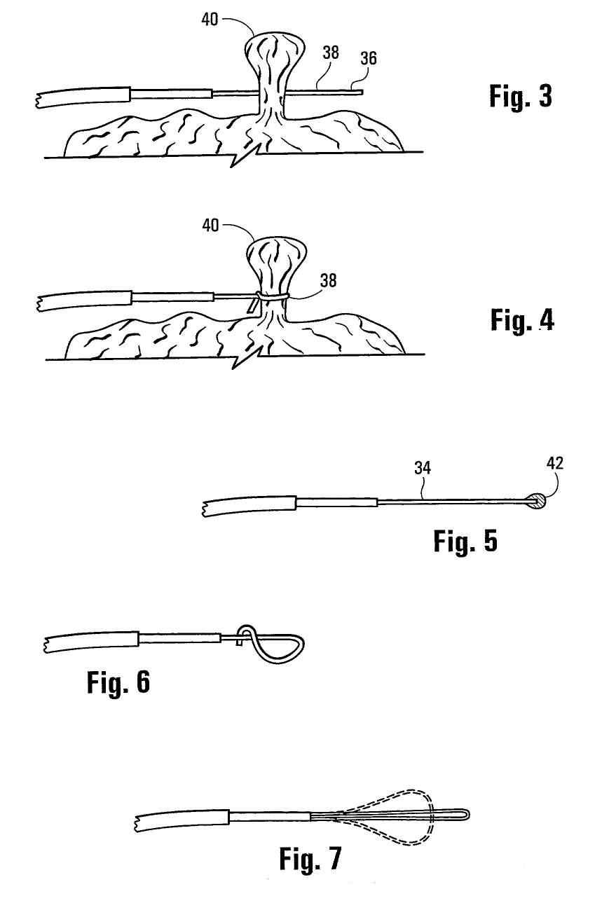

In use, a surgeon places the working end 32 of the probe into the patient's body with the assistance of an endoscope. The endoscope allows the surgeon to locate the polyp or growth designated for removal, and allows the user to view the movement of the working end 32 of surgical tool 100 inside the patient's body. As can be seen in FIG. 3, once the polyp or growth designated for removal has been located, the surgeon maneuvers filament 34, while in its straight working position 36, next to the base of polyp 40.

Once shape memory filament 34 has been positioned next to polyp 40, the surgeon applies an electric current to filament 34 through probe 22 (from the power supply connected to terminal 18). The electric current heats filament 34 and activates its pretreated shape memory position so that it transitions to curved working position 38. As can be seen in FIG. 4, when the shape memory properties of filament 34 are activated by heat, filament 34 reverts to its pretreated state, thereby encircling the base of polyp 40. In a non-limiting example of implementation, at this point the electrical current can be turned off until the surgeon is ready to excise the polyp. Alternatively, the polyp can be excised directly after filament 34 encircles polyp 40 by maintaining the electrical current at the applied level, or by increasing the applied electrical current, depending on the heat required to excise the polyp or growth.

If the surgeon wishes to reposition filament 34 around the polyp, filament 34 can be retracted back into sheath 20 in order to re-straighten filament 34. The mechanical force generated by sheath 20 is sufficient to at least partially straighten filament 34 for a second try. In a non-limiting example of implementation, filament 34 can be designed to return to its initial straight position when the electrical current is stopped.

Once the surgeon is ready to excise the polyp, the electric current is re-established up to the desired intensity. The user then moves finger portion 14 of hand assembly 10 towards thumb hold 24 so that a force is applied to filament 34 that combined with the electric current, cuts through the base of polyp 40 and cauterizes the tissue.

It should be understood that it is within the scope of the invention for filament 34 to have a straight working position and a plurality of curved working positions. For example, in a non-limiting example of implementation, filament 34 has a first curved working position that is a large continuous loop, and a second curved working position that is a small tight loop. When a first level of electrical current is applied through filament 34 such that the temperature of filament 34 elevates to a first temperature, filament 34 forms into the first working position. When the level of electrical current applied to filament 34 is increased such that the temperature of filament 34 elevates to a second temperature that is higher than the first temperature, filament 34 forms the second working position.

It will be appreciated that several variations of the configuration of filament 34 can be envisioned. In an alternate embodiment shown in FIG. 5, filament 34 includes a blob of a bio-compatible substance 42 on its tip, that rounds out the potentially sharp tip of filament 34. Thereby making the tip blunt. Therefore, the blob of bio-compatible substance 42 prevents filament 34 from inadvertently spearing the tissue during insertion, thereby making it easier to maneuver during surgery.

In a further specific embodiment, as seen in FIG. 6, filament 34 can be pre-formed to have a hook 44 at its tip. In this embodiment hook 44 can latch onto the lower portion of filament 34 upon formation of its curved working position, thereby preventing filament 34 from unwinding as the surgeon pulls on probe 22.

In yet another embodiment, as seen in FIG. 7, filament 34 may be in the shape of a flattened loop with a rounded loop shape memory position. In this configuration, when an electric current is applied, filament 34 opens up to form a loop of a pre-formed shape, which in this case is a rounded loop as shown by the dotted lines. A loop formed into the shape of a square, triangle, rectangle, or any other shape is also within the scope of the present invention. In addition, a kit which would allow a surgeon or technician to “train” filament 34 into a particular shape prior to surgery may be provided. Such a kit would allow a user to form filament 34 into any unique shape that would best suit the needs of a particular patient.

In yet another embodiment of surgical tool 100, two filaments of shape memory alloy are attached to probe 22 so that the two filaments wrap around polyp 44 simultaneously upon being heated by an electric current. This embodiment provides increased strength to surgical tool 100, for removing larger and thicker polyps. Alternatively, additional strength can be added to surgical tool 100 by using a flat piece of shape memory alloy wire that has a width and a thickness, wherein the width is greater than the thickness.

The above description of preferred embodiments should not be interpreted in a limiting manner since other variations, modifications and refinements are possible within the spirit and scope of the present invention. The scope of the invention is defined in the appended claims and their equivalents.

What is claimed is:

1. A surgical tool for removing a growth from within a patient's body, said surgical tool comprising:
an electrically conductive probe having a working end adapted to be inserted into a patient's body;
a shape memory alloy filament located at said working end and having a first working position and a second working position, wherein;
a) in said first working position, said shape memory alloy filament is in a condition that enables it to be inserted into a patient's body and placed next to the growth;
b) in said second working position, said shape memory alloy filament forms a bend around the growth;
said shape memory alloy filament transitioning from said first working position to said second working position in response to the passage of electrical current therethrough that elevates a temperature of said shape memory alloy filament ;
said shape memory alloy filament when in said second working position around the growth, and when hot, operative to excise the growth.

2. A surgical tool as defined in claim 1, wherein the growth is a polyp.

3. A surgical tool as defined in claim 2, wherein in said first working position said shape memory alloy filament is substantially straight.

4. A surgical tool as defined in claim 3, wherein in said second working position said shape memory alloy filament encircles the growth.

5. A surgical tool as defined in claim 4, wherein said shape memory alloy filament is a Ni—Ti wire with a diameter of 0.015 inches.

6. A surgical tool as defined in claim 1, wherein said shape memory alloy filament is flat and has a width and a thickness, wherein said width is greater than said thickness.

7. A surgical tool as defined in claim 1 further comprising a handle assembly that includes:
a terminal for connection to an electrical current supply unit;
a frame portion having a thumb hold and a track, and;
a finger portion that is operable to move on said track in relation to said frame portion between a retracted position and an extended position.

8. A surgical tool as defined in claim 7, wherein an electrically insulating sheath is attached to one of said frame portion and said finger portion.

9. A surgical tool as defined in claim 8, wherein said electrically conductive probe is attached to the other one of said frame portion and said finger portion, and is slidably housed within said electrically insulating sheath.

10. A surgical tool as defined in claim 9, wherein when said finger portion is in said retracted position, said electrically conductive probe is in a retracted position with respect to said sheath.

11. A surgical tool as defined in claim 10, wherein when said finger portion is in said extended position, said electrically conductive probe is in an extended position with respect to said sheath.

12. A surgical tool as defined in claim 11, wherein said shape memory alloy filament includes a tip with an amount of bio-compatible substance positioned thereon.

13. A surgical tool as defined in claim 12, wherein said tip is blunt.

14. A process for removing a growth from within a patient's body, said process comprising:
providing a shape memory alloy filament having a first working position and a second working position, wherein;
a) in said first working position, said shape memory alloy filament is in a condition that enables it to be inserted into a patient's body and placed next to the growth;
b) in said second working position, said shape memory alloy filament forms a bend around the growth;
c) said shape memory alloy filament transitioning from said first working position to said second working position in response to the passage of electrical current therethrough that elevates a temperature of said shape memory alloy filament;
d) said shape memory alloy filament when in said second working position around the growth, and when hot, operative to excise the growth;
inserting said shape memory alloy filament in into the patient's body while said shape memory alloy filament is in said first working position;
positioning said shape memory alloy filament while in said first working position next to the growth;
applying an electrical current through said shape memory alloy filament for transitioning said shape memory alloy filament into the second working position wherein said shape memory alloy filament forms a bend around the growth;
excising the growth with the shape memory alloy filament.

15. A method for setting at least one working position of a shape memory alloy filament for use in excising a growth from within a patient's body, said method comprising:
forming the shape memory alloy filament into a bend;
heating the shaped memory alloy filament while in the formed bend;
quenching the shaped memory alloy filament while in the formed bend.

16. A method as defined in claim 15, wherein the shape memory alloy filament is quenched by immersing the shape memory alloy in water.
